(19) United States
(12) Patent Application Publication
(10) Pub. No.: US 2015/0359570 A1
(43) Pub. Date: Dec. 17, 2015

(54) SURGICAL KIT FOR PLACING AN ACCESS TUBE IN THE INTERVERTEBRAL DISK OF A PATIENT

(71) Applicant: JOIMAX GMBH, Karlsruhe (DE)

(72) Inventor: Wolfgang RIES, Linkenheim (DE)

(73) Assignee: JOIMAX GMBH, Karlsruhe (DE)

(21) Appl. No.: 14/765,170

(22) PCT Filed: Jul. 3, 2014
(86) PCT No.: PCT/EP2014/001834

§ 371 Date: Jul. 31, 2015

(30) Foreign Application Priority Data
Aug. 14, 2013 (DE) 20 2013 007 340.3

(51) Int. Cl. A61B 17/70; A61M 25/06; A61M 25/09; A61B 17/00; A61B 17/34

(52) U.S. Cl. CPC A61B 17/7061; A61B 17/00234; A61B 17/7074; A61B 17/3421; A61B 17/3417; A61M 25/09; A61M 25/0662; A61B 2017/00261; A61B 2218/002

(57) ABSTRACT

A surgical kit for placing an access tube (4) in the intervertebral disc of a patient, including a stylet (1), a cannula (2), a guiding wire (3) and an access tube (4) to be placed. In order to create access to the surgical site in a way that is easy and less stressful for the patient, an obturator (5) is provided for the access tube (4), the outside diameter of which corresponds to the inside diameter of the access tube (4) and which has a lumen of a diameter that corresponds to the diameter of the guiding wire (3).

### CROSS REFERENCE TO RELATED APPLICATIONS

This application is a United States National Phase application of International Application PCT/EP2014/001834 filed Jul. 3, 2014 and claims the benefit of priority under 35 U.S.C. §119 of German Utility Model Application DE 20 2013 007 340.3 filed Aug. 14, 2013, the entire contents of which are incorporated herein by reference.

### FIELD OF THE INVENTION

The present invention pertains to a surgical kit providing access to an intervertebral disk for placing an access tube into the intervertebral disk of a patient, with a stylet, a cannula, a guide wire and a guide tube to be placed.

### BACKGROUND OF THE INVENTION

An access tube, through which further instruments and tools are inserted for observing the surgical site and for performing the corresponding surgical activities, is to be placed first in case of minimally invasive percutaneous surgical procedures (i.e., surgical procedures performed through the skin of a patient), especially endoscopic surgical procedures in the area of the vertebral canal of a patient, such as the removal of an intervertebral disk, obliteration of tissue, processing of areas of vertebral bodies, etc.

A unit comprising a (hollow) cannula and a stylet located therein, both of which are pointed on their distal end faces, are usually inserted for this up to the center of the intervertebral disk through an incision in the skin. The stylet is subsequently removed from the cannula and a guide wire is pushed through this [cannula] with its distal end up to the opposite margin of the anulus fibrosis, and the hollow cannula, which has a Luer adapter design at its proximal end for connection with the stylet that is at first located in it, is then pulled out via the guide wire and removed. Furthermore, dilation of the access tube is then performed from the skin surface to the vertebral canal by means of sleeve-like dilators by pushing first a dilator with a very small diameter over the guide wire, then over this a dilator with a larger diameter, etc., pushing in a total of two to four dilators. After pushing in the last dilator, the dilators located in this as well as the guide wire are removed and an access tube for instruments and tools, preferably in the form of an irrigation shaft, is pushed in through the dilator with the largest diameter, which is left in place.

The above description shows that dilation of the access tube is performed according to this method in the radial direction beyond a cross section that is necessary for the access tube to be finally provided or the irrigation shaft, because the latter (the access tube) is passed, as was stated, through the indwelling obturator, so that the latter must have a larger diameter than the access tube or the irrigation shaft. This leads to greater traumatization and damage to the tissue than these are absolutely necessary for performing the procedure.

### SUMMARY OF THE INVENTION

A basic object of the present invention is to create a surgical kit for creating an access to the intervertebral disk, especially for placing an access tube to the vertebral canal, which makes it possible to place the access tube in a simpler and less complicated manner, faster and consequently in a more time-saving manner as well as with less traumatic stress for the patient.

This object is accomplished according to the present invention with an intervertebral disk surgical kit of the type mentioned in the introduction, which is characterized by an obturator for the access tube, whose external diameter corresponds to the internal diameter of the access tube and which has a lumen whose diameter corresponds to the diameter of the guide wire.

It becomes possible due to the present invention, after removal of the cannula over the guide wire pushed in through this, so that only the guide wire is in place from the skin surface to the intervertebral disk, to push in the unit comprising the access tube and the obturator located in this together to the surgical site at the intervertebral disk and to bring about in one step a dilation of the access path in the patient's tissue, and for a preset diameter of the access tube, dilation must be performed to a smaller diameter and it is therefore less traumatic, because no dilator (guide sleeve) surrounding the access tube for pushing in is necessary.

After pushing in the unit comprising the access tube and the cannulated obturator, the latter are removed together with the guide wire, so that only the access tube, which is preferably designed as an irrigation shaft, may remain in place. Irrigation of the surgical site can then be performed through this access tube, and additional instruments and tools can be inserted, as this was described in the introduction.

The oblong, especially cylindrical and rod-shaped parts of the kit consist of metal, preferably (stainless) steel, and nitrate may be considered for use as well. The guide wire is preferably designed as a massive part, especially as a one-piece rod, and proximal structural elements of one component, such as the adapter head and grip parts preferably consist of plastic, such as polyethylene (PE) and polypropylene (PP).

While the other instrument parts have usual dimensions, especially the guide wire and the wall of the access tube, which wall is made as thin as possible (0.15 mm to 30 mm), as this is required by the requirements imposed in terms of stability and especially rigidity, the obturator has a greater wall thickness than it is necessary for static reasons and for reasons of stability between its inner, central axial lumen and its outer wall. In particular, at least the wall of the obturator is at least as thick as the diameter of the guide wire, so that the external diameter of the obturator is consequently three times the thickness of the guide wire. As a result, a considerable dilation of the access canal can be performed in one step through the patient's tissue from his skin surface to the vertebral canal during the insertion of the unit comprising the access tube and the obturator located in this.

Provisions are made in a preferred embodiment for a distal end area of the obturator to taper toward the distal end face of the obturator. The access tube should be rigid especially over more than three quarters of its length. It is extremely preferable if the entire access tube is rigid. Provisions are made in another preferred embodiment for the distal end face of the obturator to be blunt. The obturator has a lumen with an opening on its distal end face. The fact that the end face is blunt means that the wall surrounding the opening, especially the end-face ring area thereof, is blunt, and has, above all, no cutting elements, such as blades or cutting edges. The ring-shaped end face may be rounded or flattened. The risk of injury to tissue that is to be passed through is reduced hereby. In addition, the dilation of the tissue to be passed through takes place more gently.

To support a less stressful dilation of the access path in the patient's tissue, provisions are made in further embodiments of the present invention for the distal end area of the obturator to taper towards the distal end face thereof (distal end face of the obturator), in which case the tapering of the obturator may be made especially convexly conical, and the tapering especially preferably takes place parabolically in the longitudinal section from the outer side to the central axis of the obturator with the origin of the parabolic shape being located on the axis of the obturator.

Provisions are made in further embodiments of the present invention for the tapering distal end area of the obturator to project over the distal end of the access tube in the distal direction.

Provisions are made in another preferred embodiment, with the obturator inserted maximally into the access tube, for the distal end face of the access tube to coincide axially with the transition of the obturator from the cylindrical main part thereof with constant diameter to the tapered area thereof. The maximum depth to which the obturator is inserted into the access tube is limited by proximal stop parts of both elements, such as a head part of the guide wire and a grip part on the obturator. The tissue is dilated especially gently during the insertion due to such a continuous transition tapering in the distal direction without edges. Moreover, provisions may be made for the external diameter of the stylet to correspond to the internal diameter of the hollow cannula and/or for the stylet to be able to be connected with a connection head of the hollow cannula in an axially rigid and torque-proof manner. In particular, the connection may be designed as a Luer adapter.

Moreover, provisions may be made for the access tube to be an irrigation shaft, wherein especially the access tube has an irrigation port at its proximal end. The irrigation port is located laterally from the access tube acting as an irrigation shaft, preferably at an angle of 60° to 90°.

A procedure is made possible by the surgical kit according to the present invention such that a cannula with a stylet inserted into it is passed through the skin of the patient up to the vertebral canal, the stylet is subsequently removed from the cannula and a guide wire is pushed in through the cannula, and the cannula is removed thereafter, after which a unit comprising the access tube to be placed and an obturator located in this with an axial lumen is pushed in by means of the lumen of the obturator over the guide tube up to the vertebral canal while dilating the access path in the patient's body, and the obturator and the guide wire are subsequently removed from the access tube.

It is possible to proceed especially such that the dilation of the guide path through the patient's tissue is performed up to the intervertebral disk by means of a tapering end area of the obturator and/or that the surgical site of the patient located distally from the distal end of the access tube is irrigated proximally at the access tube designed as an irrigation shaft after pushing in the access tube and removing the obturator with the guide wire. Provisions are made in further procedures for inserting further instruments and/or tools, such as endoscopes, forceps, grippers, cutters and high-frequency probes, up to the surgical site located distally from the distal end of the access tube through the access tube after removal of the obturator and guide wire from the access tube.

Provisions are made in preferred embodiments for the lumen of the obturator to have a diameter of 2 mm to 5 mm, for the wall thickness of the access tube to be between 0.25 mm and 0.5 mm and/or for the external diameter of the cannula to be between 0.9 mm and 1.2 mm, wherein a cannula with an external diameter of 0.9 mm to 1.0 mm (according to EN ISO 4626) is called a 20G cannula (G=gauge) and a cannula with an external diameter of 1.2 mm is called an 18G cannula.

Provisions are made in a preferred embodiment for the surgical kit to comprise exclusively the aforementioned parts.

The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

In the drawings:

FIG. 1 is a view of a hollow cannula with a stylet located in the hollow cannula;

FIG. 1a is an enlarged view of a distal end area of the hollow cannula and the inserted stylet;

FIG. 1b is an enlarged view of the distal end area of the hollow cannula and the inserted stylet in an alternative embodiment;

FIG. 2 is a view of the hollow cannula with a guide wire passed through the hollow cannula after removal of the stylet;

FIG. 3 is a view of a bare guide wire after removal of the hollow cannula;

FIG. 4 is a view of a unit comprising an irrigation shaft as an access tube with an obturator located in this, which unit is pushed in via the guide wire up to the vertebral canal;

FIG. 5 is a view illustrating a joint removal of the obturator and the guide wire;

FIG. 6 is a view of the irrigation shaft remaining in a surgical position as an access tube;

FIG. 7 is a schematic view of an obturator alone; and

FIG. 8 is a longitudinal sectional view of the obturator.

### DESCRIPTION OF THE PREFERRED EMBODIMENTS

The surgical kit or set according to the present invention comprises basically the following parts: stylet 1, hollow cannula 2, guide wire 3, access tube 4 in the form of an irrigation shaft and obturator 5.

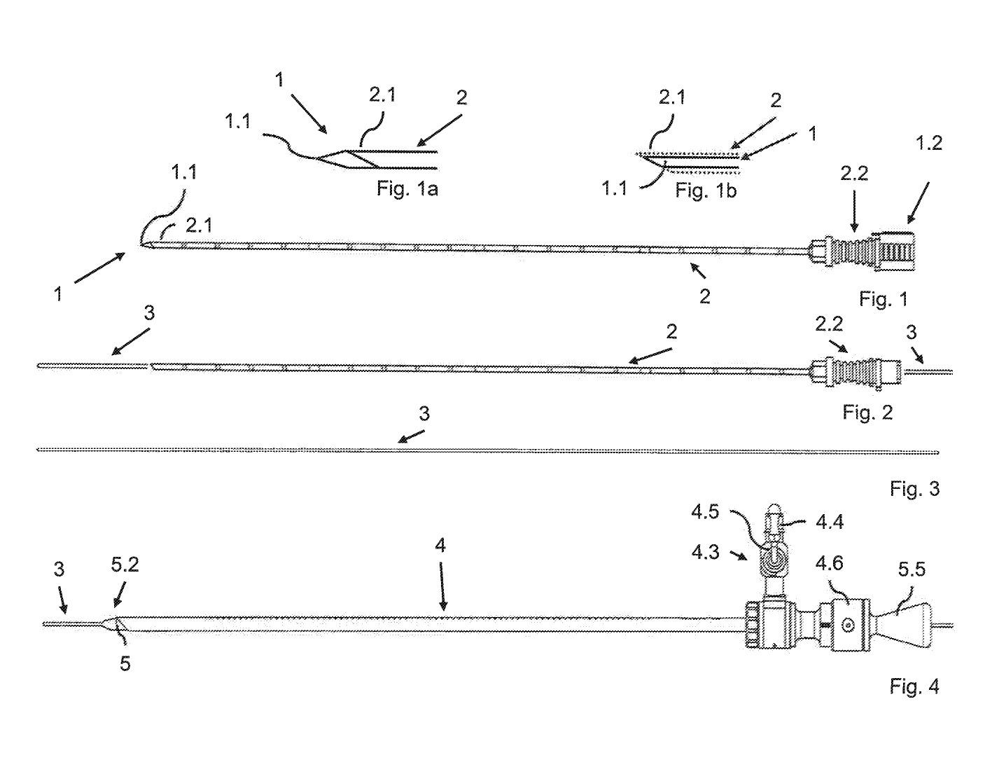

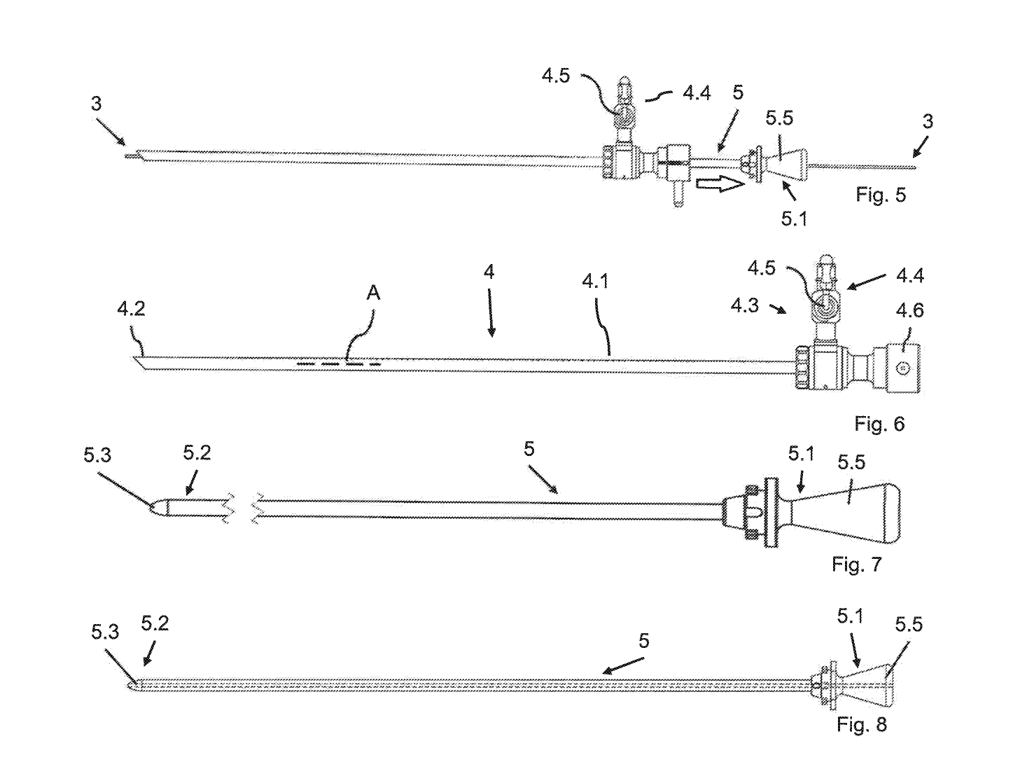

The stylet is shown in FIG. 1 in its position in which it is inserted into the hollow cannula 2 only, and only the distal tip 1.1 of the stylet and the connection head 1.2 thereof can be recognized. FIG. 1a shows this situation in an enlarged form.

The hollow cannula 2 is designed as a cutting cannula and has a tip 2.1 beveled on one side at its distal end. It is likewise provided with a connection head 2.2 at this proximal end, and the connection heads 1.2 and 2.2 are designed as Luer adapters, so that the hollow cannula 2 and the stylet 1 can be connected with one another for insertion especially in an axially rigid manner, but also such that they rotate in a torque-proof manner.

FIG. 1b shows an alternative embodiment of the stylet 1 and the hollow cannula 2, in which the distal tip 1.1 of the stylet 1 is made flush with the tip 2.1 of the hollow cannula, and the end face of the stylet 1 and the hollow cannula 2 extends flatly with a finite angle not equal to 90° in relation to the axes of the parts 1, 2 over the entire cross-sectional area of both parts and forms especially an angle of 30° to 65° with the axis.

The external diameter of the stylet 1 corresponds to the internal diameter of the hollow cannula 2. This means that the two standard diameters are basically equal and the diameters are adapted, taking tolerances into account, such that the stylet 1 can be inserted into and passed through the hollow cannula 2 and then removed from the hollow cannula 2 without major difficulties and without overcoming major frictional forces, without needless radial intermediate spaces being formed between the outer circumference of the stylet 1 and the inner circumference of the hollow cannula 2.

The gauge of the guide wire is maximally such that it likewise corresponds to the above dimensioning rules in respect to the lumen of the hollow cannula 2. The guide wire may also be made thinner, so that it can be inserted through the hollow cannula 2 with a clearance. At any rate, the length of the guide wire 3 substantially projects over the length of the hollow cannula 2 (and hence also that of the stylet 1), at least by one quarter, preferably by one third of the length of the hollow cannula 2. Both head parts of the guide wire 3 are made blunt and as conical sections.

The essential new parts of the surgical kit or set according to the present invention are the parts shown in FIG. 4, namely, the access tube 4 and the obturator 5. The same dimensioning rules apply qualitatively as they were described above with respect to the hollow cannula 2 and the stylet 1 concerning the internal diameter of the access tube 4 and the external diameter of the obturator 5, on the one hand, and concerning the internal diameter of the lumen of the obturator 5 and the external diameter of the guide wire 3 used.

The longitudinal dimensions of the access tube, especially of the access tube 4, especially of the tube shaft 4.1 proper, are on the same order of magnitude as those of the hollow cannula 2, and preferably somewhat larger. The distal end face 4.2 of the access tube 4 is likewise beveled on one side, i.e., with an end-face bevel extending continuously from one side of the wall of the tube shaft 4.1 to the diametrically opposite side, e.g., at an angle of 45° in relation to the axis A of the longitudinal extension of the access tube 4. At the proximal end, the access tube 4 has a head part 4.3, which will be described in more detail below.

The obturator 5 has such a length that when it has been pushed completely into and passed through the access tube 4 with the stop of its head part 5.1 at the adapter part 4.6 of the head part 4.3 of the access tube 4, it protrudes with its distal end area 5.2 from the end face 4.2 of the access tube 4. The distal end area 5.2 of the obturator 5 tapers towards the distal end face 5.3. While a conical design of the tip is basically possible, the taper has, preferably and in the exemplary embodiment being shown, a convex design, whose radius of curvature is smaller on the end face 5.3 than at the transition located proximally hereto to the cylindrical shaft part of the obturator 5, so that the outer wall of the tapering distal end area 5.2 follows essentially a parabolic shape with its origin on the distal end face 5.3 in the section from the central axis A to the lateral wall of the obturator 5.

The obturator 5 has a grip part 5.5 at the proximal end of its head part 5.1. Together with the adapter part 4.6 of the head part 4.3 of the access tube 4, a stop is formed, which limits the maximum depth of penetration of the obturator 5 into the access tube 4. The distal end face 5.3 of the obturator projects in this position over the distal end face 4.2 of the access tube, doing so such that a nearly continuous, edgeless transition is formed from the tapering distal end face 5.3 of the obturator 5 to the distal end face 4.2 of the access tube 4.

The grip part 5.5 has two pins, which come to lie axially behind undercut edges of the adapter part 4.6 of the access tube in the state in which the obturator 5 has maximally penetrated into the access tube 4 in the direction in which the obturator 5 extends. These edges are radially offset about the axis along which the access tube 4 extends, so that a relative axial motion of the access tube 4 and the obturator 5 is possible. In the state of maximum penetration, a lever (not shown) arranged on the adapter part 4.6 makes possible an axial rotation of the access tube 4 and obturator 5. As a result, the pins of the grip part 5.5 extend behind the undercut edges of the adapter part 4.6 and block in this state a relative axial motion of the two parts. This locking is released again by rotating the lever in the opposite direction.

The head part 4.3 of the access tube 4 has a radially extending lateral irrigation port 4.4, via which the surgical site located at the distal end face 4.2 of the access tube 4 in the area of the intervertebral disk can be irrigated from the outside of the patient's body via the irrigation port 4.4 and the shaft 4.1 of the access tube 4, so that the access tube 4 is designed as an irrigation shaft in the exemplary embodiment being shown.

The irrigation port is provided, further, with a valve lever 4.5. This has a conical shape and is fixed at the opposite end to the irrigation port 4.4. The valve lever 4.5 has a hole-like bore, which is aligned flush with the interior space of the irrigation port 4.4 in the opened state. The bore is oriented at right angles to this direction in the closed state, so that the access to the access tube is blocked.

The surgical technique for inserting the access tube 4 through the skin of a patient up to the surgical site in the vertebral canal is as follows:

The unit comprising the hollow cannula 2 and the inserted stylet 1 is first inserted through an incision into the patient's skin up to the vertebral canal.

While loosening the Luer adapter formed by the parts 1.2 and 2.2 by rotating the parts against each other, the stylet 1 is subsequently removed from the hollow cannula 2.

The guide wire 3 is then pushed into and through the hollow cannula until the distal end of the guide wire 3 reaches the surgical site.

The hollow cannula 2 is then pulled off in a further procedural step in the proximal direction over the guide wire 3, so that only the guide wire 3 is left in place at first, as this is shown in FIG. 3.

The unit comprising the access tube 4 and the inserted obturator projecting over the access tube with its distal end area is subsequently pushed into the surgical site, and the tapering distal end area 5.2 of the obturator 5 or the distal end area 5.2 expanding from the distal end face 5.3 of the obturator to the circumference of the cylindrically shaped shaft part 5.4 of the obturator brings about a dilation of the passage channel, which will be kept open by the access tube 4 in the further course.

The obturator 5 is removed via the grip part 5.5 together with the guide wire 3 from the access tube 4 in a last step.

Irrigation of the surgical site can then take place, and an endoscope can be subsequently pushed in through the access tube 4 in order to inspect the surgical site through this and the optical system thereof in front of the distal end 4.2 of the access tube 4. Furthermore, usual surgical procedures, such as the insertion of cutting, gripping or milling tools through the access tube 4 or even the pushing in of a high-frequency probe through the access tube 4 may be performed in order to obliterate tissue parts in the area of the vertebral canal or to perform denervation.

While specific embodiments of the invention have been shown and described in detail to illustrate the application of the principles of the invention, it will be understood that the invention may be embodied otherwise without departing from such principles.

1. An intervertebral disk access surgical kit for placing an access tube to an intervertebral disk of a patient, the intervertebral disk access surgical kit comprising:
a stylet;
a cannula;
a guide wire comprising a guide wire diameter;
an access tube comprising an access tube internal diameter; and
an obturator for the access tube, said obturator comprising an obturator external diameter, said obturator external diameter corresponding to said access tube internal diameter, said obturator comprising a lumen, said lumen comprising a lumen diameter, said lumen diameter corresponding to the guide wire diameter.

2. A kit in accordance with claim 1, wherein the access tube comprises a proximal end and a length, said access tube being rigid over at least a majority of said length from said proximal end.

3. A kit in accordance with claim 1, wherein said obturator comprises a distal end area and a distal end face, said distal end area tapering toward said distal end face.

4. A kit in accordance with claim 3, wherein a taper of the obturator has a convex arc-shaped design.

5. A kit in accordance with claim 4, wherein the taper is parabolic with an origin of a parabolic shape located on a central axis of the obturator in a longitudinal section from an outer side to the central axis of the obturator.

6. A kit in accordance with claim 3, wherein the distal end face of the obturator is blunt.

7. A kit in accordance with claim 1, wherein a tapering distal end area of the obturator projects over a distal end of the access tube in a distal direction.

8. A kit in accordance with claim 1, wherein with the obturator inserted maximally into the access tube, a distal end face of the access tube axially coincides with a transition of the obturator from a cylindrical main part of the obturator with constant diameter to a tapered area of the obturator.

9. A kit in accordance with claim 1, wherein the access tube has a distal end, a one-sided bevel and a tip as a distal end face at said distal end.

10. A kit in accordance with claim 1, wherein the access tube is designed as an irrigation shaft.

11. A kit in accordance with claim 10, wherein the access tube comprises a proximal end, the access tube having an irrigation port at said proximal end.

12. A kit in accordance with claim 11, wherein the irrigation port is provided laterally from the access tube.

13. A kit in accordance with claim 12, wherein the irrigation port is directed at an angle of 90° to an axis of the access tube.

14. A kit in accordance with claim 1, wherein an external diameter of the stylet corresponds to an internal diameter of the hollow cannula.

15. A kit in accordance with claim 1, wherein the stylet is connected with a connection head of the hollow cannula via a connection heed in an axially rigid and torque-proof manner.

16. A kit in accordance with claim 1, wherein the lumen diameter is 2 mm to 5 mm.

17. A kit in accordance with claim wherein a wall thickness of the access tube is between 0.25 mm and 0.5 mm.

18. A kit in accordance with claim 1, wherein an external diameter of the cannula is between 0.9 mm and 1.2 mm.

19. A kit in accordance with claim 1, wherein the kit comprises exclusively the stylet, the cannula, the guide wire, the access tube and the obturator.

20. A method for placing an access tube into an intervertebral disk of a patient, the method comprising:
passing a cannula with an inserted stylet through skin of the patient to the intervertebral disk, wherein the stylet is subsequently removed from the cannula and a guide wire is pushed in through the cannula, and the cannula is then removed;
providing a unit comprising the access tube to be placed and an obturator with an axial lumen, said obturator being located in the access tube;
pushing in the unit up to a center of the intervertebral disk by means of the lumen of the obturator via the access tube while dilating an access path in a body of the patient and the obturator and the guide wire are subsequently removed from the access tube.

21. A method in accordance with claim 20, wherein dilation of the access path through tissue of the patient up to the intervertebral disk is performed by means of a tapering end area of the obturator.

22. A method in accordance with claim 21, wherein the dilation of the access path through the tissue of the patient is performed by means of an end area expanding convexly from an end face of the obturator to a cylindrical part of said obturator.

23. A method in accordance with claim 20, wherein after pushing in the access tube and removing the obturator with the guide wire, a surgical site of the patient located distally from a distal end of the access tube is irrigated at an irrigation port arranged proximally at an end of the access tube, the irrigation port being arranged at the access tube designed as an irrigation shaft.

24. A method in accordance with claim 20, wherein after removal of the obturator and the guide wire from the access tube, one or more of additional instruments and tools are inserted through said access tube up to a surgical site located distally from a distal end of the access tube.
